(19) United States
(12) Patent Application Publication
(10) Pub. No.: US 2021/0289802 A1
(43) Pub. Date: Sep. 23, 2021

(54) ENZYME PRODUCT WITH BETA-GALACTOSIDASE

(71) Applicant: WeissBioTech GmbH, Ascheberg (DE)

(72) Inventors: Christian NAUMER, Mannheim (DE); Christian ZUREK, Lorsch (DE); Ümit PUL, Duisburg (DE)

(73) Assignee: WeissBioTech GmbH, Ascheberg (DE)

(21) Appl. No.: 16/622,959

(22) PCT Filed: Jun. 15, 2018
(86) PCT No.: PCT/EP2018/065962

§ 371 Date: Dec. 16, 2019

(30) Foreign Application Priority Data
Jun. 16, 2017 (DE) 20 2017 103 594.8

(51) Int. Cl. A23C 9/12; C12N 9/38

(52) U.S. Cl. CPC A23C 9/1206; A23C 9/1216; C12Y 302/01023; C12Y 301/06001; C12N 9/2471

(57) ABSTRACT

The invention relates to an enzyme product comprising a beta-galactosidase product. The invention provides that the beta-galactosidase product has an inactive arylsulphatase.

### SEQUENCE LISTING STATEMENT

The ASCII file, entitled 79024_ST25.txt, was created on Jun. 9, 2021 and comprises 3,398,723 bytes.

### DESCRIPTION

The present invention relates to an enzyme product comprising a beta-galactosidase product according to independent claim 1 and an enzyme product obtainable by a neutral beta-galactosidase according to independent claim 6.

### State of the Art

The invention relates to the production of a lactase for the manufacture of lactose-free products which have neither a disturbing taste nor smell.

Enzymatic processes have been used for centuries in the production of food. One of the oldest of these enzymatic production processes is the production of cheese from milk, which would not be possible without the enzyme chymosin extracted from the stomachs of cattle calves. The enzyme chymosin, also called lab, is called coagulation enzyme.

Coagulation enzymes or coagulants have also been discovered in the stomachs of other mammals but also in microorganisms such as Rhizumucor mihei, Rhizomucor pussilus or Cryphonectria parasitica. In the meantime, coagulation enzymes from mammals are produced using microbial production strains such as fungi, e.g. Aspergillus oryzae, or yeasts, e.g. Kluyveromyces lactis (K. lactis).

Over the years, more and more enzymes have found use in the production of dairy products. Lysozyme from chicken eggs is used as an antimicrobial protection. Phospholipases improve the yield in cheese production as well as the taste of the cheese. Lipases of microbial origin are used to improve the taste of cheese. Enzymes are also used to produce lactobionic acid from lactose.

Lactases are particularly important additives for the milk processing industry. Lactases or beta-galactosidases (EC 3.2.1.108) are enzymes that break down lactose, a disaccharide, into its monosaccharides, glucose and galactose. Lactases are used both to produce certain dairy products, such as Dulce de Leche, and to remove lactose from milk.

Treatment with lactase can remove lactose from milk. The resulting lactose-free milk products can be consumed by lactose intolerant consumers without the unpleasant side effects.

Lactases are either used as a process aid in the manufacture of lactose-free dairy products or are taken by lactose intolerant consumers before consumption of a dairy product in tablet form or as a powder. In addition to being used for dietary reasons, lactase is also used to improve dairy products.

Depending on the type of application, there are lactases that are used in the neutral pH range for pure dairy products or lactases with an acidic activity range for the production of e.g. whey-based products.

All industrial lactases are of microbial origin. The neutral lactases are produced from yeasts such as Kluyveromyces lactis (K. lactis) or Kluyveromyces marxianus (K. marxianus), while the acid lactases are obtained from Aspergillus spec.

Some of the lactase products from yeasts have a side activity that can give the lactase-treated dairy product an unpleasant odor. This secondary activity is the activity of the enzyme arylsulphatase (EC 3.1.6.1), which cleaves cresol sulfate into cresol and sulfate. Cresol is the cause of the unpleasant smell.

In EP 1 954 808 B1 a lactase product containing a small amount of arylsulphatase is revealed.

### DISCLOSURE OF THE INVENTION

The object of the present invention is to overcome the above-mentioned disadvantages at least in part. In particular, the object of the present invention is to create lactase products that are pleasant to use.

The foregoing object is solved by an enzyme product having the characteristics of claim 1 and an enzyme product obtainable by a neutral beta-galactosidase having the characteristics of Claim 6.

Further characteristics and details of the invention result from the dependent claims, the description, the drawings as well as the mentioned examples. Characteristics and details which have been described in connection with the enzyme product in accordance with the invention shall, of course, also apply in connection with the enzyme product in accordance with the invention obtainable by a neutral beta-galactosidase as well as the use in accordance with the invention and the method in accordance with the invention and vice versa in each case, so that with regard to the disclosure of the individual aspects of the invention mutual reference is or can always be made.

An enzyme product according to the invention for the production of a beta-galactosidase product is characterized by the fact that the beta-galactosidase product contains an inactivated arylsulphatase. Arylsulphatase is an enzyme which occurs naturally in yeasts and is contained as a beta-galactosidase preparation by-product. The release of p-cresol in milk creates an unpleasant smell and taste. The inactivation can take place genetically. During genetic inactivation, special genes are regulated, in particular downregulated, so that gene expression is inactivated. In particular, the expression of a gene can be influenced or inactivated by manipulating the start of transcription or translation. The start of transcription, in particular at promoters, can be altered by genetic regulation of the specific DNA sequence and thus the expression of the entire gene can be activated, inhibited or inactivated. It is advantageous that an inactivated arylsulphatase does not produce any arylsulphatase at all, in particular no arylsulphatase gene is expressed at all, so that a disturbing odor and/or taste is not only reduced, but is not present at all.

Furthermore, in the context of the present invention, it is conceivable that the enzyme product according to the invention comprises a beta-galactosidase product containing a lactase of K. lactis. Not every enzyme product from yeasts of the genus Kluyveromyces contains arylsulphatase. By classical strain development or the selection of a suitable production strain it is possible to produce an arylsulphatase-free beta-galactosidase product. By combining classical genetic modifications or, as in this example, by “genome editing”, the production of arylsulphatase can be inhibited by specifically switching off the arylsulphatase gene in order to enable the production of an arylsulphatase-free beta-galactosidase product. With an arylsulfatase-free beta-galactosidase product, no unpleasant odor and/or taste resulting from the activity of the enzyme arylsulphatase can be produced in the milk. Furthermore, it is conceivable that the invention is directed at a K. lactis strain for the production of an enzyme product according to the invention, wherein the K. lactis strain has an inactivated, in particular genetically inactivated, arylsulphatase. It is advantageous that the method according to the invention can be performed in K. lactis.

Furthermore, it is possible within the scope of this invention that the gene for arylsulphatase is inactivated by means of genome editing methods using artificial nucleases (zinc finger nuclease, ZFN; Transcription Activator-Like Effector Nuclease, TALEN) or programmable nucleases of CRISPR technology, in particular CRISPR-Cas9. CRISPR technology is based on the components of widespread prokaryotic immune systems, which are summarized under the acronym CRISPR (clustered regularly interspaced short palindromic repeats). CRISPR cassettes are regions in the prokaryotic genomes that consist of an accumulation of short, often palindromic, DNA sequence repeats. The cas (CRISPR-associated) genes coding for Cas proteins with nucleic acid-binding, helicase and/or nuclease activities are located in the immediate vicinity of the CRISPR cassettes. The components of the prokaryotic CRISPR systems, in particular Class II systems, are used for genome editing. These include Cas proteins with nuclease activity, in particular Cas9, Cpf1 or other class II CRISPR nucleases, whose double-stranded DNA sequence specificities can be modulated with short CRISPR RNAs (crRNAs) or synthetic single-guide RNAs (sgRNAs). By using CRISPR technology, the arylsulphatase gene can be inactivated. Thus, this gene is no longer completely/sensitively readable, the arylsulphatase is not produced functionally and a corresponding smell/flavor can no longer be produced in milk products treated with beta-galactosidase.

Furthermore, it is conceivable within the scope of the present invention that the beta-galactosidase product contains a lactase from K. lactis 21B7, K. lactis 21B7ΔAS #1 and/or K. lactis 21B7ΔAS #9 (CBS deposit numbers: CBS 142344 for K. lactis 21B7, CBS 142345 for K. lactis 21B7ΔAS #1 and CBS 142346 for K. lactis 21B7ΔAS #9). For K. lactis 21B7 the strain K. lactis Y1118 (CBS 6315) is the precursor strain. For K. lactis 21B7ΔAS #1 and K. lactis 21B7ΔAS #9, K. lactis 21B7 is the precursor strain. Both strains K. lactis 21B7ΔAS #1 and K. lactis 21B7ΔAS #9 are preferably produced by the CRISPR/Cas method. All strains are deposited in the CBS strain collection and contain no foreign DNA or a new combination of genetic material. The strains comprising only the deletion of a single base pair (K. lactis 21B7ΔAS #9) or the deletion of 289 base pairs (K. lactis 21B7ΔAS #1) in the arylsulphatase gene.

According to another aspect of the invention, the enzyme product is available through a neutral beta-galactosidase from K. lactis to produce an arylsulphatase-free beta-galactosidase product. The use according to the invention has the same advantages as described in detail with regard to the composition according to the invention. As is the case here, inactivation can take place particularly at the genetic level. Specific genes can be regulated, in particular downregulated. Or in another form the gene is still expressed, but the gene product is formed in an inactive form. In both cases it prevents the formation of a disturbing smell and/or taste.

According to another aspect of the invention, the beta-galactosidase product is a product for the treatment of dairy products. Consequently, the present invention also covers foodstuffs, in particular milk products containing the enzyme product of the invention. It is advantageous that this can be milk, especially cow's milk, or milk from other livestock such as sheep or goats. Furthermore, the beta-galactosidase product may be used for the treatment of one of the following dairy products: Fresh milk, UHT milk, buttermilk, cream fraiche, soured milk, condensed milk, yoghurt, yoghurt product, cream, cream product, sour cream, mixed milk product, cream product, butter, milk powder, cheese, cheese product, curd, Dulce de Leche. Furthermore, the invention is aimed at cosmetic products as well as cleaning and/or care products with the invention enzyme product, which have the described advantages.

Furthermore, it is advantageous if the enzyme product according to the invention, the arylsulphatase-free beta-galactosidase product, is produced from production strains optimized by the application of artificial or programmable nucleases, in particular CRISPR technology. Advantageously, the CRISPR technology can be used to inactivate the arylsulphatase gene and thus the function of the gene product. Thus, this gene is no longer functional and a corresponding smell and/or taste can no longer be formed during the production of lactase products.

The invention-based enzyme product is obtainable in particular by a neutral lactase from Kluyveromyces lactis, for the production of a beta-galactosidase product. The beta-galactosidase product is essentially free of arylsulphatase activity. By essentially free it can be understood that the beta-galactosidase product has only minor to no arylsulphatase activity, so that the described benefits are achieved within the scope of the enzyme product comprising a beta-galactosidase product.

According to another aspect of the invention, the product of a neutral beta-galactosidase from K. lactis is used to produce an arylsulphatase-free beta-galactosidase product. The use according to the invention has the same advantages as described in detail with regard to the composition according to the invention.

According to a further aspect of the invention, a method for preparing a beta-galactosidase product is comprised, wherein the beta-galactosidase product contains an inactive arylsulphatase.

The method for preparing a beta-galactosidase product may advantageously comprise at least one of the following steps:
a) Determination of the target sequence of the gene to be inactivated in the production strain, preferably K. lactis 21B7,
b) Construction of a vector containing Cas9 and an sgRNA specific for the arylsulphatase gene of the beta-galactosidase-producing strain, preferably K. lactis 21B7,
c) Transformation of the beta-galactosidase-producing strain, preferably K. lactis 21B7,
d) Sequencing of the target sequence in the transformants to verify the successful deletion or introduction of individual nucleotides.
e) Removal of the vector from (b) knock-out mutants produced/confirmed by cultivation under non-selective conditions,
f) Permeabilization of the knock-out mutants by chemical treatment,
g) Separation of the permeate from the biomass,
h) Determination of the enzyme activities of beta-galactosidase and arylsulphatase in permeate (ratio arylsulphatase units/neutral lactase units, ASU/NLU).

The individual steps do not necessarily have to be performed in the specified order.

Molecular Biological Methods

The Escherichia coli strains (E. coli) DH5-alpha and DH10-beta serve as recipient organisms for the construction and propagation of CRISPR/Cas9 [pKmARS7] vector derivatives. The cultivation of produced E. coli strains takes place in Luria-Bertani medium (LB medium) containing 5 g/l yeast extract, 10 g/l tryptone, 5 g/l sodium chloride. For use as a solid medium, LB medium is mixed with 1.5% (w/v) agar. All media are heat sterilized before use. Where selective cultivation conditions are required, the media contain ampicillin at a final concentration of 100 μg/ml and kanamycin at a final concentration of 25 μg/ml.

The genesis of the beta-galactosidase producer K. lactis 21B7 is described in the work of Wellenbeck et al., Engineering in Life Sciences (2016), doi:10.1002/elsc.201600031. The strain is usually cultivated in complex Yeast Peptone Dextrose Medium (YPD Medium) containing 10 g/l yeast extract, 10 g/l peptone and 20 g/l glucose. For use as a solid medium, YPD medium is mixed with 2.0% (w/v) agar. All media are heat sterilized before use. Recombinant K. lactis 21B7 cells with introduced [pKmARS7] vector derivatives are cultured in YPD medium with the antibiotic geneticin (G418) at a final concentration of 25 μg/ml.

Commercially prepared chemically and electrically competent E. coli DH5-alpha or DH10-beta cells are purchased from New England Biolabs (NEB, Frankfurt) for transformation purposes and treated according to the manufacturer's specifications. Transformed E. coli cells are identified under selective conditions on LB-Agarmmedien.

Constructed [pKmARS7] vectors for targeting the genomic arylsulphatase gene sequence are introduced by electroporation into K. lactis 21B7 cells. The preparation of the electrocompetent cells and the implementation of the transformation are performed according to a published method by Sanchez et al., Applied and Environmental Microbiology 59 (1993), 2087-92. 10 ml YPD medium are inoculated with a single colony and incubated for 16 h at 30° C. on a horizontal shaker with a frequency of 250 rpm. Using cells from this culture, 100 ml of YPD medium are subsequently adjusted to an optical density of 0.2 at 600 nm (OD600). The inoculated culture shall be incubated on a horizontal shaker with a frequency of 250 rpm until an optical density OD600 of 0.8 to 1.4 at 30° C. is reached. After harvesting the culture by centrifugation for 5 min at 10° C. and 1900×g, the cell pellet obtained is converted into 10 ml pretreatment buffer [YPD medium containing 20 mM 4-(2-hydroxyethyl)-piperazine-1-ethanesulfonic acid, N-(2-hydroxyethyl)-piperazine-N′-2-ethanesulfonic acid (HEPES), pH 8.0; 25 mM 1,4-Dithioerythritol (DTT)] and incubated for 30 min at 30° C. on a horizontal shaker with a frequency of 100 rpm. Separate the pretreated cells from the suspension by centrifugation for 5 min at 10° C. and 1900×g and resuspend the obtained cell pellet in 10 ml electroporation buffer [10 mM Tris-HCl, pH 7.5; 270 mM sucrose; 1 mM lithium acetate]. After a further centrifugation step for 5 min at 10° C. and 1900×g, the pelletized cells are finally taken up in 300 μl electroporation buffer. Cell aliquots of 50 μl are stored at −80° C. For the transformation, 50 μl cells with 300 ng plasmid DNA are mixed in an electroporation cell with a slit width of 2 mm and stored on ice for 15 minutes. Electroporation is performed in a BioRad Gene Pulser Xcell electroporation system (BioRad, Munich) at 1.0 kV, 400Ω and 25 mF. Immediately after the pulse, the cells in the cuvette are first diluted in 1 ml ice-cold YPD medium, then transferred to a conical 15 ml tube and cultured for 4 hours at 30° C. on a horizontal shaker with a frequency of 250 rpm. Transformed K. lactis 21B7 cells with introduced [pKmARS7] vector derivatives are selected on geneticin-containing YPD agarmed medium for 2 to 5 days at 30° C.

The methods used for the isolation of plasmid DNA, the enzymatic modification of nucleic acids, the ligation of nucleic acids, the enzymatic amplification of nucleic acids by polymerase chain reaction (PCR) and the electrophoretic separation and purification of nucleic acids in agarose gels are state-of-the-art. Unless otherwise noted, the methods are performed according to the protocols described in the standard work of Sambrook et al. Molecular Cloning, A Laboratory Manual, 2nd edition 1989, CHS Press, Cold Spring Harbor.

The GeneJET Plasmid Miniprep Kit (Thermo Fisher Scientific, Darmstadt) is used for the isolation of plasmid DNA from recombinant E. coli cells according to manufacturer specifications. The amplification of nucleic acids by PCR is preferably performed with the Phusion Flash High-Fidelity PCR System (Thermo Fisher Scientific, Darmstadt). All oligonucleotides used were synthesised by the service provider biomers.net (biomers.net, Ulm). If necessary, nucleic acids from enzymatically catalyzed reactions and agarose gels are purified with the Qiagen MinElute System (Qiagen, Hilden) or the ZymoClean Gel DNA Recovery System (Zymo Research, Freiburg) according to manufacturer protocols. The identity of generated PCR amplicons and cloned nucleic acids will be determined by Sanger sequencing at the service provider GATC Biotech (GATC Biotech, Constance).

The isolation of genomic DNA from the strain K. lactis 21B7 is carried out from 2 OD cells (OD600) of an overnight culture with the Zymo Research YeaStar Genomic DNA System (Zymo Research, Freiburg). Degradation of the cell wall with the enzyme zymolyase is performed for 1 h at 37° C. and the purified genomic DNA is eluted into 60 μl Qiagen EB buffer (Qiagen, Hilden).

Analysis of the Target Sequence in K. lactis 21B7

The genome sequence of the strain K. lactis NRRL Y-1140 available under the access number NC_006042 (Seq ID NO: 20) in the NCBI database (www.ncbi.nlm.nih.gov/nucleotide/) serves as a reference for amplification and sequence analysis of the arylsulphatase gene in K. lactis 21B7. In the genome of the strain K. lactis NRRL Y-1140 the annotated open reading frame KLLA0_F03146g and a size of 1668 bp (Seq ID NO: 21) encodes a conserved hypothetical protein with 555 amino acids (Seq ID NO: 22). The Kyoto Encyclopedia of Genes and Genomes database (KEGG, http://www.genome.jp) assigns the protein the function of an arylsulphatase (KLLA0F03146g) based on sequence homology. The experimental proof for the arylsulphatase activity of the protein can be shown in the work of Stressler et al., Applied Microbiology and Biotechnology, 100 (2016), 5401-5414. The derived primers ATM358 (5′-TTCGCTCCACCCGATCATGAGAGTTTACAG-3′ Seq ID NO: 01) and ATM359 (5′-TGTAATGGCCAATGTGGAGCTGTGTAGGTC-3′ Seq ID NO: 02) generate in a PCR reaction with genomic DNA from K. lactis 21B7 is a specific amplicon having a size of 894 bp (PCR conditions: 30 ng genomic DNA, 12.5 pmol primer; 25 μl reaction volume; initial denaturation, 98° C., 10 sec; PCR cycle: 35×[98° C., 5 sec; 60° C. 5 sec; 72° C., 14 sec], 72° C., 60 sec). The amplicon generated is composed of 238 bp of the 5′-UTR and 656 bp of the coding arylsulphatase gene sequence. To determine the nucleotide sequence, the blunt end PCR products are cloned into the Thermo Fisher Scientific pCR blunt vector system (Thermo Fisher Scientific, Darmstadt) according to the manufacturer's protocol and the ligation products are transformed into competent E. coli cells. Plasmid DNA is isolated from recombinant E. coli cells and the sequence of the inserted amplicons is determined by Sanger sequencing with the primers M13f (5′-TGTAAAACGACGGCCAGT-3′ Seq ID NO: 03) and M13r (5′-CAGGAAACAGCTATGACC-3′ Seq ID NO: 04) flanking the cloning site. The nucleotide sequence determined for the 894 bp amplicon is shown in sequence 1. Compared to the reference sequence from K. lactis NRRL Y-1140, a base difference in the coding arylsulphatase gene sequence can be observed for the corresponding genome segment in K. lactis 21B7. The change of the triplet from CGT to AGT in the nucleic acid sequence (sequence 1) leads to the exchange of the amino acid arginine for serine in the primary sequence at position 139 of the protein (sequence 2). According to the publication of Stressler et al., Applied Microbiology and Biotechnology, 100 (2016), 9053-9067, the protein with the amino acid serine in position 139 represents the active arylsulphatase variant. The sequence information obtained serves as a reference for the inactivation of the arylsulphatase gene in K. lactis 21B7 by CRISPR/Cas9.

Sequence 1 comprises an 894 bp amplicon (primer pair ATM358/ATM359) with the partial arylsulphatase sequence from K. lactis 21B7 (in brackets: open reading frame, ORF):

Seq ID NO: 05
TTCGCTCCACCCGATCATGAGAGTTTACAGAAAGTTCTTACCTTGAAACTT
TTTTACTGCACCCTTTTTATCACTTCGAGTAAAGCTTTAAATGACATAATT
TACAATATAAATATGGATCATCTCACCTGTCATCATGGTCTTTTAGGAATT
GTGAAGGTATACTAACCAATTGTTACTCTATGATCTATCCGAAAATCCGCA
AACTGCATTAGAGAAAAGTACAATCGAATTGCA[ATGACCAAAACAGATGA
ACCTAAAAAGCCGAATTTCTTAATTATTGTCGCCGATGATTTAGGGTTTAC
AGATGTGTCGAGTTTTGGAGGTGAAATACAAACGCCAAACCTTGATAAATT
GTCTAAAGGTGGTTTCAGATTCACTGGTTTTCATACTGCATCGGCATGTTC
GCCAACTAGATCGATGTTGTTGAGCGGTACTGACAACCATTTGGCAGGTTT
GGGCCAAATGGCTGAATATGCCAGGCAATTTCCAGAAAAGTTCAAGGATAA
ACCTGGTTATGAAGGTTATTTGAACTATAAGGTTGCTGCATTGCCTGAAAT
ATTATCCCCAGAATATTACACGCTGATTTCGGGGAAATGGCACCTTGGATT
AGAAAAGCCATATTGGCCAAGTGACCGTGGATTCCAAAAAAGTTTTACCTT
GCTGCCTGGTGCAGGAAACCATTTCAAATGCAATTTGGATAGCAAGTTTCT
TCTCCCATGGATTTATCAGGAAAATGGCGAAAGGGTAGACCATAACAAGTT
TCCAGAAAACTTTTATTCCACCACTTACTTCACAGATAAATTTTTGGAATA
TTTGAAGGATGATGAAGAAAGAAAAGGTCGCCCATTCTTTGGCCAGTTGAC
CTACACAGCTCCACATTGGCCATTACA].

Sequence 2 comprises a translated protein partial sequence of arylsulphatase from K. lactis 21B7:

Seq ID NO: 06
MTKTDEPKKPNFLIIVADDLGFTDVSSFGGEIQTPNLDKLSKGGFRFTGFH
TASACSPTRSLLSGTDNHLAGLGQMAEYARQFPEKFKDKPGYEGYLNYKVA
ALPEILSPEYYTLISGKWHLGLEKPYWPSDRGFQK[S]FTLLPGAGNHFKC
NLDSKFLLPWIYQENGERVDHNKFPENFYSTTYFTDKFLEYLKDDEERKGR
PFFGQLTYTAPHWPL

Construction of the [pKmARS7] Base Vector

The base vector used with a size of 3924 bp contains at least one of the following elements: ARS7 replicon, TEF1p promoter, EM7p promoter, CYC1t transcription terminator, kanMX resistance gene, ColE1 replicon, CPS1t transcription terminator. The element ARS7 is an autonomously replicating DNA sequence ARS7 from K. marxianus DSM70344 (Autonomously Replicating Sequence, KmARS7) for the episomal propagation of the vector in K. lactis 21B7. The element TEF1p promoter is a promoter of the Transcription Elongation Factor 1 gene from Saccharomyces cerevisiae (S. cerevisiae) for the expression of the kanamycin resistance gene in K. lactis 21B7. The element EM7p promoter is a synthetic prokaryotic promoter for the expression of the kanamycin resistance gene in E. coli. The element CYC1t transcription terminator is a 3′ region to the CYC1 gene from S. cerevisiae for terminating the transcription of the kanMX resistance gene. The element kanMX resistance gene is a kanamycin resistance gene. The element ColE1 replicon is used for episomal replication of the vector in E. coli. The element CPS1t transcription terminator is a 3′ region to the CPS1 gene from S. cerevisiae for the termination of inserted genes in the flanking cloning site.

Genome Editing

The application of CRISPR/Cas9 technology for the genome editing of selected genes in K. lactis is exemplarily shown in the work of Horwitz et al., Cell Systems 1 (2015), 88-96. The expression of the human codon-optimized Cas9 nuclease and the transcription of base-specific single-guide RNA (sgRNA) enables the precise introduction of DNA double-strand breaks in K. lactis ATCC 8585 and subsequently the inactivation or alteration of target genes via the cell's own repair mechanism via non-homologous end joining (NHEJ). For the introduction of knock-out mutations into the gnomically encoded arylsulphatase gene in K. lactis 21B7, a CRISPR/Cas9 system was established based on the present E. coli/K. marxianus shuttle vector [pKmARS7]-MCS2-CPS1T-kanMX.

A SNR52 transcription cassette was inserted into the base vector [pKmARS7] (see above) with the hCas9 expression cassette ([pKmARS7]-TPI1p-hCas9-CPS1t-kanMX) to generate the chimeric sgRNA consisting of the crRNA and tracrRNA sequences. The principle structure of sgRNA corresponds to the construction published in the work of Jinek et al., Science 337 (2012), 816-821. The 426 bp DNA fragment comprising downstream of the SNR52 RNA polymerase III promoter (DiCarlo et al., Nucleic Acids Res 41 (2013), 4336-4343), a BsmBI cloning site for the modulation of the first 20 nucleotides (target specific guide sequence) of sgRNA by Golden Gate assembly (Engler and Marillonnet, Methods Mol Biol (2014), 119-131). For cloning purposes, the recognition sequences of the restriction endonucleases AgeI and SbfI flank the SNR52-sgRNA transcription unit. After AgeI/SbfI hydrolysis, the 423 bp cassette was inserted into the 8703 bp XmaI/SbfI [pKmARS7]-TPI1p-hCas9-CPS1t-kanMX vector fragment. Subsequently, the recognition sequences of the restriction endonuclease BsmBI in the coding hCas9 and kanMX gene sequences were removed in the generated vector by introducing synonymous triplets. The modification of these sequences allows the direct introduction of target specific guide sequences into the BsmBI hydrolyzed vector [pKmARS7]-TP11p-hCas9-SNR52p-sg RNA.

Further measures to improve the invention result from the following description of some embodiments of the invention. All characteristics and/or advantages resulting from the claims or the description, including all details, possible uses and method steps, may be essential to the invention, both in themselves and in various combinations. It should be noted that the examples are only descriptive and are not intended to restrict the invention in any way.

### Example 1: Inactivation of Arylsulphatase in K. lactis 21B7 by CRISPR/Cas9

The nucleotide sequence of arylsulphatase in the genome of strain K. lactis 21B7 (see sequence 1) serves as a reference for the identification of potential Cas9 target sequences in the coding gene sequence of the enzyme. The base sequence “N₍₂₀₎NGG” Seq ID NO: 07 (Deltcheva et al., Nature 471 (2011), 602-607) is regarded as a suitable target sequence for the activity of the SpCas9 nuclease. The canonical “NGG” sequence motif is referred to as “Protospacer adjacent motif (PAM)” and immediately follows the 20-base target sequence (Protospacer). The activity of the Cas9-sgRNA complex leads to a DNA double strand break at a defined position located three base pairs upstream of the PAM within the protospacer.

For the genetic inactivation of arylsulphatase in K. lactis 21B7, the following three CRISPR/Cas9 target sequences with the base sequence “N₍₂₀₎NGG” Seq ID NO: 07 in the corresponding coding gene sequence are identified as examples. The PAM motifs are shown bold in the sequences.

Protospacer PAM Target Sequence No. 1:

Bases 266 to 288 in sequence 1, non-codogenic DNA strand,

Seq ID NO: 08
5′-GCGACAATAATTAAGAAATTCGG-3′

Protospacer PAM Target Sequence No. 2:

Bases 344 to 366 in sequence 1, codogenic DNA strand

Seq ID NO: 09
5′-AACCTTGATAAATTGTCTAAAGG-3′

Protospacer PAM Target Sequence No. 3:

Bases 430 to 452 in reference sequence No. 1, codogenic DNA strand,

Seq ID NO: 10
5′-GAGCGGTACTGACAACCATTTGG-3′

The molecular biological procedure for the genetic inactivation of arylsulphatase in the genome of strain K. lactis 21B7 is illustrated below using the identified Protospacer-PAM target sequence No. 1 as an example: The complementary oligonucleotides ATM381_Sp1AS_f (5′-GATCGCGACAATAATTAAGAAATT-3′ Seq ID NO: 11) and ATM382_Sp1AS_r (5′-AAACAATTTCTTAATTATTGTCGC-3′ Seq ID NO: 12) are inserted to the BsmBI-hydrolyzed CRISPR/Cas9 vector [pKmARS7]-TPI1p-hCas9-SNR52p-sgRNA by Golden Gate assembly and the ligation products into competent E. coli cells. The identity of the inserted oligonucleotides was confirmed in isolated plasmid DNA by Sanger sequencing. The generated construct [pKmARS7]-TPI1p-hCas9-SNR52p-Sp1ASsgRNA is introduced by electroporation into competent K. lactis 21B7 cells and transformants on YPD-agarmedium with geneticin (G418) are selected. The genomic DNA of selected recombinant clones serves as a PCR template for the detection of potential IndeI mutations (insertion/deletion) in the selected CRISPR/Cas9 target sequence. For this purpose, the primers ATM358 (5′-TTCGCTGCCACCCGATCATGAGTTTTACAG-3′ Seq ID NO: 01) and ATM359 (5′-TGTAATGCCAATGTGGTGAGCTGTAGGTAGGTC-3 Seq ID NO: 02 ‘) are used to generate a specific PCR-product and determining the nucleotide sequence of the amplicon in the region of the CRISPR/Cas9 targeting site with the primer ATM397 (5’-TGGAATCCACGGTCACTTGG-3′ Seq ID NO: 13) by Sanger sequencing. Sequence analysis leads to the detection of a base pair deletion in the area of the selected protospacer sequence for the majority of the clones investigated. The generated frameshift in position 33 of the coding arylsulphatase gene sequence causes the premature termination of the reading frame. The genetic inactivation of the target gene by CRISPR/Cas9 is shown in sequence 3 for the clone #9 K. lactis 21B7 Sp1AS #9. Sequence 4 shows the resulting ORF and the corresponding protein sequence of arylsulphatase.

Sequence 3 comprises a deletion mutant K. lactis 21B7 Sp1AS #9 (partial sequence of the coding arylsulphatase gene). The deleted nucleotide is shown in brackets:

Seq ID NO: 14
ATGACCAAAACAGATGAACCTAAAAAGCCGAA[T]TTCTTAATTATTGTCG
CCGATGATTTGGGTTTACAGATGTGTCGAGTTTTGGAGGTGAAATACAAAC
GCCAAACCTTGATAAATTGTCTAAAGGTGGTTTCAGATTCACTGGTTTTCA
TACTGCATCGGCATGTTCGCCAACTAGATCGATGTTGTTGAGCGGTACTGA
CAACCATTTGGCAGGTTTGGGCCAAATGGCTGAATATGCCAGGCAATTTCC
AGAAAAGTTCAAGGATAAACCTGGTTATGAAGGTTATTTGAACTATAAGGT
TGCTGCATTGCCTGAAATATTATCCCCAGAATATTACACGCTGATTTCGGG
GAAATGGCACCTTGGATTAGAAAAGCCATATTGGCCAAGTGACCGTGGATT
CCA.

Sequence 4 comprises a resulting ORF of 39 bp and the associated protein sequence of arylsulphatase in clone K. lactis 21B7 Sp1AS #9: ATG ACC AAA ACA GAT GAA CCT AAA AAG CCG AAT TCT TAA Seq ID NO: 15 (translated protein sequence: MTKTDEPKKPNS Seq ID NO: 16).

In the analyzed clone #1 K. lactis 21B7 Sp1AS #1 the deletion of 289 bp in the arylsulphatase gene can be detected as a consequence of the Cas9-sgRNA activity with protospacer No. 1. The deleted area is shown in sequence 5. Sequence 6 shows the resulting ORF and the corresponding protein sequence of arylsulphatase for the deletion mutant K. lactis 21B7 Sp1AS #1.

Sequence 5 comprises a deletion mutant K. lactis 21B7 Sp1AS #1 (N-terminal partial sequence of the coding arylsulphatase gene, the region in brackets corresponds to the deletion region of strain CBS 142345):

Seq ID NO: 17
ATGACCAAAACAGATGAACCTAAAAAGCCGAAT[TTCTTAATTATTGTCGC
CGATGATTTAGGGTTTACAGATGTGTCGAGTTTTGGAGGTGAAATACAAAC
GCCAAACCTTGATAAATGTCTAAAGGTGGTTTCAGATTCACTGGTTTTCAT
ACTGCATCGGCATGTTCGCCAACTAGATCGATGTTGTTGAGCGGTACTGAC
AACCATTTGGCAGGTTTGGGCCAAATGGCTGAATATGCCAGGCAATTTCCA
GAAAAGTTCAAGGATAAACCTGGTTATGAAGGTTATTTGAACTATAAGGTT
GCTGCATTGCCTGAAA]TATTATCCCCAGAATATTACACGCTGATTTCGGG
GAAATGGCACCTTGGATTAGAAAAGCCATATTGGCCAAGTGACCGTGGATT
CCA.

Sequence 6 comprises a resulting ORF with 60 nucleotides and associated protein sequence of arylsulphatase in clone K. lactis 21B7 Sp1AS #1: ATGACCAAAACAGATGAACCTAAAAAGCCGAATTATTATCCCCAGAATATTACACGCTGA Seq ID NO: 18, translated protein sequence: MTKTDEPKKPNYYPQNITR Seq ID NO: 19.

The CRISPR/Cas9 [pKmARS7] vector derivatives used are removed from the characterized K. lactis 21B7 arylsulphatase knock-out mutants by multiple cultivation passages in YPD medium without antibiotic addition.

### Example 2: Expression of Lactase and Arylsulphatase in K. lactis 21B7 and Production of K. lactis Biomass

Biomass of the K. lactis strain 21B7 (CBS 142344) and the deletion mutants CBS 142345 and CBS 142346 with intracellular lactase is increased by fermentation. This can be achieved by cultivation for 72 hours in a mineral salt medium suitable for yeasts and feeding a substrate feed using state-of-the-art fermentation methods. Examples of this are documented in numerous publications (Wellenbeck et al., Engineering in Life Sciences (2016); Inchaurrondo et al., Process Biochemistry (1994); Fonseca et al., Applied Microbiology and Biotechnology (2013)).

The produced biomass contains the intracellular target enzyme lactase. In order to enrich this, the biomass is first broken down. The cell disruption methods described by Fenton et al. (1980) are used for this purpose, in which lactase is released into the aqueous phase by contact of the cells with an alkyl alcohol or a dialkyl alcohol. Afterwards the cells are separated by filtration and the enzyme is concentrated by ultrafiltration at a cut-off of 50 kDa to an activity of about 7500 NLU (Neutral Lactase Units)/g and purified by diafiltration with a phosphate buffer. The lactase preparation is then stabilized by the addition of glycerol corresponding to 25% of the total volume.

The amount of lactase contained in the preparations is measured in Neutral Lactase Units (NLU) of enzyme activity. One NLU corresponds to the conversion of 1.3 μmol substrate per minute under the conditions of the assay. In the assay used, the synthetic substrate ortho-nitrophenol-beta-D-galactopyranoside (ONPG) is hydrolytically cleaved from the lactase and the products ortho-nitrophenol (ONP) and galactose are formed. The reaction takes place over a period of 15 min at 30° C. in a 100 mM potassium phosphate buffer at pH 6.5 with 1 mM magnesium sulphate and 0.05 mM EDTA. The amount of substrate converted can be calculated from the absorption of the product ONP at 420 nm by calibration with different concentrations of ONP solutions.

The amount of arylsulphatase contained in the preparations is measured in arylsulphatase units (ASU) of enzyme activity. The activity is measured with the substrate paranitrophenylsulfate according to the measurement method according to De Swaaf et al., European Patent Application, EP2439266A2 (2012). An ASU is defined as the change in OD at 410 nm (OD410)*10E6 per hour under the conditions of the assay.

Further measures to improve the invention result from the figure shown. All features and/or advantages resulting from the claims, the description or the drawings, including design details, spatial arrangements and method steps, may be essential to the invention both in themselves and in various combinations. It should be noted that the figure is only descriptive and is not intended to restrict the invention in any way. It shows

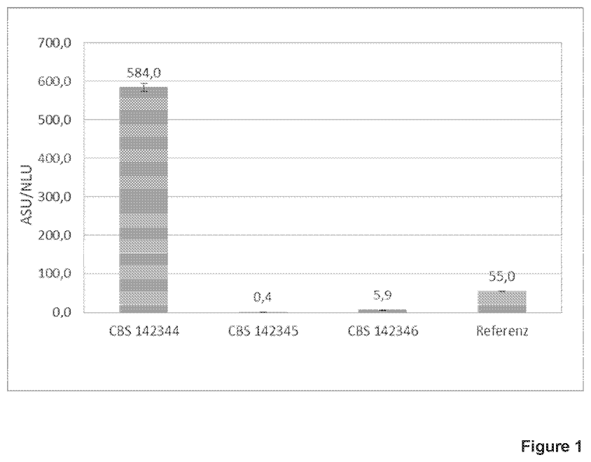

FIG. 1: Results of the quantification of the arylsulphatase activity in the lactase preparations of the investigated strains of K. lactis

FIG. 1 shows the arylsulphatase activity as a function of lactase activity (ASU per NLU). In the lactase preparation, which is produced from fermentation with the strain K. lactis 21B7, the average arylsulphatase activity is 590 ASU per NLU, with a commercially available reference enzyme 55 ASU/NLU. For the deletion mutants K. lactis 21B7 Sp1AS #1 and K. lactis 21B7 Sp1AS #9 the average arylsulphatase activity is only 0 to 6 ASU per NLU. The measured values for the mutants are close to the detection limit of the assay used. Thus, the inactivation of the coding arylsulphatase gene results in a strong reduction of the activity of the arylsulphatase in the purified lactase preparation in both investigated deletion mutants or even in an activity below the detection limit of the used assay.

1. An enzyme product comprising a beta-galactosidase product
wherein
the beta-galactosidase product comprises an inactive aryl sulfatase.

2. The enzyme product according to claim 1,
wherein
the beta-galactosidase product contains a lactase from Kluyveromyces lactis.

3. The enzyme product according to claim 1,
wherein
the beta-galactosidase product comprises a lactase from Kluyveromyces lactis 21B7.

4. The enzyme product according to claim 1,
wherein
a gene for the arylsulphatase can be inactivated by means of genome editing methods using at least artificial nucleases or programmable nucleases of the CRISPR technology.

5. The enzyme product according to claim 1
wherein
the arylsulphatase is inactivated at the genome level by genome editing methods.

6. An enzyme product obtainable by a lactase from Kluyveromyces lactis, for the preparation of a beta-galactosidase product which is substantially free of arylsulfatase activity.

7. The enzyme product according to claim 6,
wherein
the essentially arylsulphatase-free beta-galactosidase product has been produced from a production organism modified by means of genome editing technologies.

8. The enzyme product according to claim 3,
wherein
the lactase is from at least one of the deposited strains CBS142344, CBS142345 and CBS142346.

9. The enzyme product according to claim 4,
wherein
at least a zinc finger nuclease or transcription activator-like effector nuclease is used.

10. The enzyme product according to claim 5,
wherein
the arylsulphatase is inactivated by the application of the CRISPR technology with class II cas endonucleases.

11. The enzyme product according to claim 10,
wherein
the class II cas endonuclease is cas9 or cpf1.

12. An enzyme product according to claim 6,
wherein the lactase is a neutral lactase.

13. The enzyme product according to claim 7,
wherein
the genome editing technology is CRISPR-Cas9 technology.
